(19) United States
(12) Plant Patent Application Publication
(10) Pub. No.: US 2015/0067936 P1
(43) Pub. Date: Mar. 5, 2015

(54) Begonia plant named 'First Blush'

(50) ;

(71) Applicant: Terra Nova Nurseries, Inc., Canby, OR (US)

(72) Inventor: Daniel M. Heims, Portland, OR (US)

(73) Assignee: Terra Nova Nurseries, Inc., Canby, OR (US)

(21) Appl. No.: 13/987,818

(22) Filed: Sep. 5, 2013

(51) Int. Cl. A01H 5/00

(52) U.S. Cl. PLT/348

(57) ABSTRACT

A new and distinct form of Begonia plant characterized by broad leaves colored silver with a dark star-shape at the leaf base that is surrounded by a purple blush, maroon leaf backs, a bushy habit, light pink flowers in winter to early spring, tolerant of cool temperatures to 33° F., and excellent vigor.

### BOTANICAL DENOMINATION

Begonia rex-cultorum

### VARIETY DESIGNATION

‘First Blush’

### BACKGROUND OF THE INVENTION

The present invention relates to a new and distinct cultivar of Begonia, of the family Begoniaceae, and given the cultivar name, ‘First Blush’. The new cultivar originated from a cross between unknown Begonia rex-cultorum parents. This selection is uniquely characterized by:

1. broad leaves colored silver with a dark star-shape at the leaf base that is surrounded by a purple blush,

2. maroon leaf backs,

3. a bushy habit,

4. light pink flowers in winter to early spring,

5. tolerant of cool temperatures to 33° F., and

6. excellent vigor.

Compared to Begonia ‘Raspberry Swirl’, an unpatented plant, the new cultivar has leaves that are flatter and not swirled, with a larger silver area and a larger star pattern at the leaf base.

Compared to Begonia ‘Fireworks’, an unpatented plant, the new cultivar has leaves that are less rugose, darker silver colored, the star pattern is smaller and does not go into the veins to the leaf margins, and the blush is around the star pattern rather than the leaf margin.

The new variety has been reproduced only by asexual propagation (cuttings and tissue culture). Each of the progeny exhibits identical characteristics to the original plant. Asexual propagation by micropropagation as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a close up of the flowers and leaf of Begonia ‘First Blush’.

FIG. 2 shows a 1 and-a-half year old plant in a growing in a one gallon container in February in Canby, Oreg.

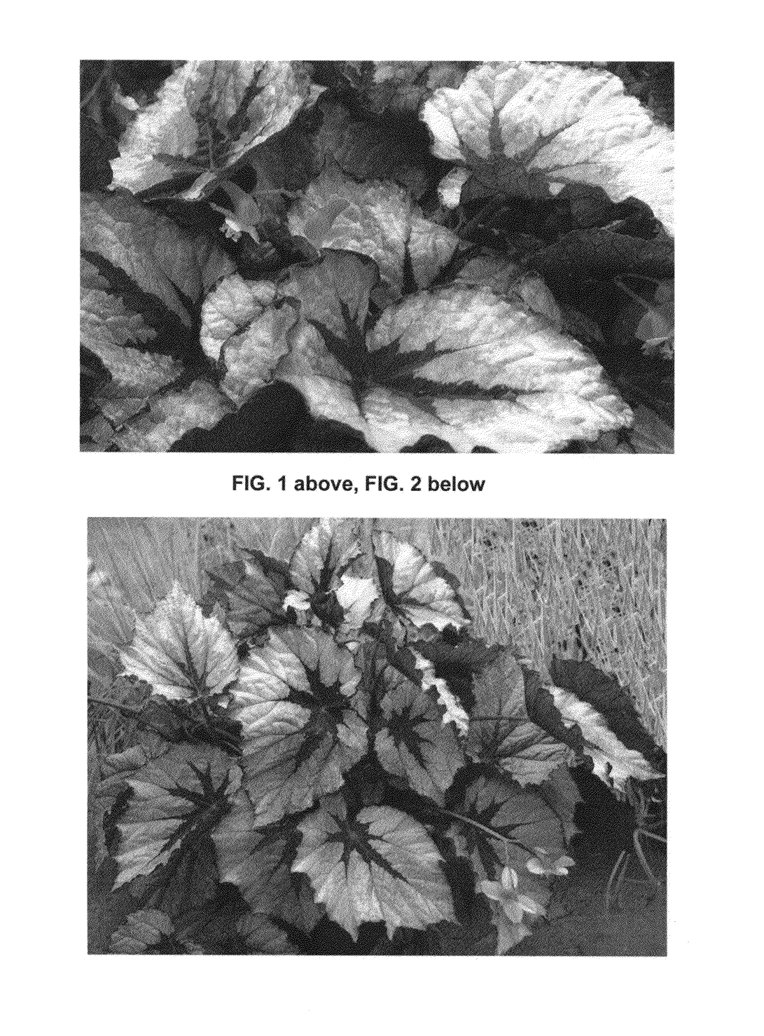

### DETAILED PLANT DESCRIPTION

The following is a detailed description of the new Begonia cultivar based on observations of nine-month-old specimens grown in one gallon containers in a cool greenhouse in early January in Canby, Oreg. Canby is Zone 8 on the USDA Hardiness map. Temperatures range from a high of 95° F. in August to 32° F. in January. Normal rainfall in Canby is 42.8 inches per year. The color descriptions are all based on The Royal Horticultural Society Colour Chart, 5^{th} edition.
Plant:
   Type.—herbaceous rhizomatous perennial.
   Form.—clumping, caulescent.
   Cold hardiness.—USDA Zone 10a.
   Size.—grows to 36 cm tall and 40 cm wide.
   Vigor.—excellent.
Stem:
   Type.—upright.
   Size.—grows to 10 cm long and 25 mm wide.
   Internodes.—from 7 mm to 1 cm long.
   Surface.—pubescent.
   Color.—Brown 200B.
Leaf:
   Type.—simple.
   Arrangement.—alternate, congested.
   Shape.—broadly ovate to orbicular.
   Margins.—undulate, coarsely serrate.
   Apex.—acute to acuminate.
   Base.—oblique.
   Venation.—palmate, Brown 200A on topside with the star pattern, bottom side Red Purple 59A.
   Blade size.—grows to 20 cm wide and 19 cm long.
   Surface texture.—pubescent on top and glabrous on bottom.
   Stipules.—deciduous, ovate-lanceolate, 30 mm long and 9 mm wide, apex acuminate and cuspidate, entire, back side pubescent, inside glabrous, both sides Red Purple 61A.
   Petiole description.—grows to 17 cm long and 8 mm wide, succulent, pubescent, Greyed Purple 187A.
   Leaf color.—top side closest to Greyed Green 188B where silver, tinted Red Purple 59B, star pattern Black 202A; bottom side Purple 59A at bottom ⅓ and the rest Greyed Green 189B heavily tinted Purple 59A.
Inflorescence:
   Type.—axillary, bisexual, cymose.
   Number of flowers per cyme.—about 4.
   Peduncle description.—grows 9 cm to 19 cm long and 2 mm to 4 mm wide, glabrous, fleshy, Greyed Purple 187C; with 2 opposite, dehiscent petiolate leaf-bracts below each cyme, which grow to 6 mm wide and 17 mm long, margins entire, tip apiculate, back side sparsely pubescent, inside glabrous, both sides Greyed Red 184C.
   Pedicel description.—grow to 25 mm long, 1.5 mm wide, fleshy, glabrous, Red Purple 63D on staminate flowers, Greyed Purple 185B on pistillate flowers.
   Bloom period.—late fall to early winter in Canby, Oreg.
Flower bud:
   Size.—staminate flowers 9 mm long and 6 mm wide prior to opening; pistillate flowers are 15 mm deep and 16 mm wide prior to opening.
   Description.—ovoid, pubescent.
   Color.—Red Purple 59D on tepal area and ovary.
Flower:
   Type.—monoecious, bilaterally symmetrical.
   Pistillate flower.—grows to 23 mm deep and 40 mm wide overall; 5 fleshy tepals, grow to 18 mm long and 16 mm wide, broadly elliptical, tip and base obtuse, margins entire, glabrous on inside and pubescent on outside, tepals Red Purple 65B on inside and 65A and 65B on outside; ovary to 30 mm wide and 12 mm deep, three winged, two equal and one longer, Greyed Purple 182A, style 3 mm long and Yellow 13B, stigma 2 to 3 branched and twisted, stigma 6 mm deep and 10 mm wide overall, Green Yellow 1C.
   Staminate flower.—12 mm deep and 33 mm wide, 4 tepals, 2 larger outer tepals 16 mm long and 15 mm wide, cupped, broadly elliptical, margin entire, tip obtuse, base truncate, Red Purple 65A inside and 63C outside; inner tepals grow to 13 mm long and 9 mm wide, oblong, entire, tip obtuse, base attenuate, both sides Red Purple 65A; stamen many in a globular head 7 mm in diameter, filament 2 mm long, anthers 1.5 mm long and Green Yellow 1D, anthers and pollen Yellow 8C.
   Fragrance.—none.
   Lastingness.—A cyme blooms for about 4 weeks on the plant.
Fruit and seed: none, sterile
Diseases and pests: The new cultivar is typical to the genus. No known resistances to pests or diseases. No problems have been found in Canby, Oreg.

I claim:

1. A new and distinct Begonia plant as hereby illustrated and described.
